(19) United States
(12) Patent Application Publication
(10) Pub. No.: US 2009/0163982 A1
(43) Pub. Date: Jun. 25, 2009

(54) APPLICATIONS OF THE STIMULATION OF NEURAL TISSUE USING LIGHT

(76) Inventor: R. Christopher deCharms, Montara, CA (US)

Correspondence Address: WILSON SONSINI GOODRICH & ROSATI, 650 PAGE MILL ROAD, PALO ALTO, CA, 94304-1050, US

(21) Appl. No.: 12/340,169

(22) Filed: Dec. 19, 2008

Related U.S. Application Data
(63) Continuation of application No. 11/281,210, filed on Nov. 15, 2005, now abandoned.
(60) Provisional application No. 60/628,258, filed on Nov. 15, 2004.

(51) Int. Cl. A61N 5/067; A61N 5/06

(52) U.S. Cl. 607/89; 607/92

(57) ABSTRACT

The present invention comprises systems and methods for stimulating target tissue comprising a light source; an implantable light conducting lead coupled to said light source; and an implantable light-emitter. The light source, lead and emitter are used to provide a light stimulation to a target tissue

### CROSS-REFERENCE

This application is a continuation of U.S. patent application Ser. No. 11/281,210, filed Nov. 15, 2005, entitled “Applications of the Stimulation of Neural Tissue Using Light,” which claims the benefit of U.S. Provisional Patent Application Ser. No. 60/628,258, filed on Nov. 15, 2004, entitled “Methods for Treating Neurological and Neurodegenerative Conditions”; each of which applications is hereby incorporated by reference in its entirety.

### FIELD OF THE INVENTION

The present invention relates generally to methods for stimulation of the nervous system.

### INCORPORATED BY REFERENCE

All publications and patent applications mentioned in this specification are herein incorporated by reference to the same extent as if each individual publication or patent application was specifically and individually indicated to be incorporated by reference. In particular, the following applications, patents, and non patent references are hereby incorporated by reference for all purposes: U.S. Publication Nos. 20030208245, 20040215286, 20020183817, 20040214790, 20030144709, and 20030181960; U.S. Pat. Nos. 6,921,413 and 5,716,377; and H. G. Sachs, et al., “Retinal Replacement—the Development of Microelectronic Retinal Prostheses—Experience with Subretinal Implants and New Aspects”, Graefe's Arch Clin Exp Opthalmol 242 (2004) 717-723, J. A. Turner, et al., “Spinal Cord Stimulation for Patients With Failed Back Surgery Syndrome or Complex regional Pain Syndrome: A Systematic Review of Effectiveness and Complications”, Pain 108 (2004) 137-147; A. M. Kuncel, et al., “Selection of Stimulus Parameters for Deep Brain Stimulation”, Clinical Neurophysiology 115 (2004) 2431-2441; M. Capecci M D, et al., “Chronic Bilateral Subthalamic Deep Brain Stimulation in a Patient with Homozygous Deletion in the Parkin Gene”, Movements Disorders Vol. 9(12) (2004) 1450-1452; and K. H. Sipson, et al., “A Randomized, Double-Blind, Crossover Study of the Use of Transcutaneous Spinal Electoanalgesia in Patients with Pain from Chronic Critical Limb Ischemia” Journal of Pain and Symptom Management Vol, 28(5) (2004) 511-516.

### BACKGROUND

Prior art has disclosed many methods of stimulating neural tissue using electricity. Recently, prior art has disclosed means of directly stimulating a peripheral nerve in an experimental preparation using a laser. The present invention discloses application of methods for stimulating target tissue using light or optical energy.

As disclosed in U.S. Pat. No. 6,921,413, upon which some aspects of this invention expand, various methods may be used to stimulate neural tissue. Several of the traditional methods of stimulation include electrical, mechanical, thermal, and chemical. A neuron will propagate an electrical impulse after applying a stimulus. The most common form of applying such stimulus is to form a transient current or voltage pulse applied through electrodes. Electrical stimulation, as well as mechanical and chemical stimulation, has many limitations. To name a few, stimulation by such methods may result in nonspecific stimulation of neurons or damage to neurons. Difficulty exists in recording electrical activity from the neuron due to an electrical artifact created by the stimulus. To stimulate only one or a few neurons, fragile micro-electrodes need to be fashioned and carefully inserted into the tissue to be stimulated. Such techniques do not easily lend themselves to implantable electrodes used for long term stimulation of neural tissue.

Fork was the first to report a direct stimulation of nerve fibers using low-energy laser light (Fork, R., “Laser stimulation of nerve cells in Aplysia”, Science, March (5): p. 907-8, 1971.) According to Fork et al., laser irradiation at (488 nm, 515 nm, and 1006 nm) was applied to the abdominal ganglion of Aplysia Californica that possesses some light sensitive properties. The author observed that the cells fired when the light at 488 nm was turned on in some cases and turned off in others. In another study, bundles of rat nervous fibers may be stimulated using a XeCl laser (Allegre, G., S. Avrillier, and D. Albe-Fessard, “Stimulation in the rat of a nerve fiber bundle by a short UV pulse from an excimer laser”, Neuroscience Letters, 180(2): p. 261-4, 1994.) When stimulated using a laser pulse transmitted through an optical fiber, a response similar to that obtained with electrical stimulation was observed. A threshold stimulation level of 0.9 J/cm² was reported for optical stimulation. No other reports by the same authors have been published since. Thus, optical energy can be used to stimulate nerve fibers. Although there is ample evidence that photon energy effects neural tissue in humans and animals, a need remains for a method that can be used to stimulate neural tissue without damaging such tissue or producing artifacts. Furthermore, in order for such an invention to be useful in both research and clinical applications, it should produce activity in neurons by delivery of energy without the addition of potentially toxic dyes or at intensities destructive to the neuron over useful periods of time. Finally, there is a need for a method of precisely stimulating an individual neuron with optical energy without piercing tissue.

One common way of providing light energy for stimulation of neural tissues is by using a laser. Lasers are characterized by their wavelength and energy level. Classically, lasers have been used in biological applications for tissue ablation. However, low power lasers are available for uses other than tissue ablation. The energy required for stimulation large populations of neurons is very small, and the energy required to stimulate an individual neuron is exceedingly small. Manipulation of strength, duration and frequency of stimulation are key parameters that determine whether a neuron will fire. Such parameters are adjustable with pulsed, optical energy and can be adjusted to a range acceptable for stimulation of neural tissue. Additionally, the precision of laser energy delivery can easily provide a novel method of selectively stimulating individual neurons or different nerve fibers within a large population of neurons without the need to pierce tissue.

The present invention provides methods for stimulating neural tissue with optical energy. Stimulation of neural tissue in this regard includes, but is not limited to, generation and propagation of an electrical impulse in one or more neurons after applying an optical stimulus. In addition, there is a unique basic science and clinical need for producing an artifact-free response in neurons that causes no damage to the tissue.

One advantage of the present invention is that the methods of stimulating neural tissue described herein may be contemplated to be highly specific to individual nerve fibers or small groups of nerve fibers. As intensity of electrical stimulation increases, progressively greater numbers of neurons are activated. This is a physical property of associated with increasing the electrical field size. Optical energy, however, can be confined to a predetermined, physical “spot” size, which is independent of the energy delivered. This physical property is what allows optical techniques to be unique in stimulation of individual or selected neurons. Another advantage of the present invention is the use of the methods of stimulation of neural tissues in vivo. In vitro methods of stimulation, on the other hand, do not lend themselves to the uses of an in vivo method.

Still another advantage of the present invention is that optical stimulation of neural tissue is not associated with an electrical stimulus artifact. Thus, when optically stimulating individual or multiple neurons stimulated by optical energy, electrical stimulus artifacts are not present.

Still another advantage of this method is that the use of low energy laser stimulation provides precise localization without tissue contact, resulting in high specificity. Such specificity is of use clinically when nerve stimulation is used for diagnostic applications like identification of subsets of peripheral nerve fibers during operative repair of severed nerves. Also, such technology would allow multiple, focused laser stimuli, to be used to provide functional mapping of neural networks and their interconnections. This advantage may also be applied in therapeutic situations such as neural modulation for pain management, control of movement disorders, and seizure reduction.

### SUMMARY OF THE INVENTION

In one embodiment, the present invention involves a system for stimulating target tissue comprising: a light source for providing stimulation pulses; an implantable light conducting lead coupled to said light source adapted for stimulation of a predetermined site in a subject. In one aspect, the light conducting lead is an optical fiber. In another aspect the light source is a laser. In one aspect, the light source is implantable.

In one embodiment, the present invention relates to a method of treating a disorder comprising: implanting at least one light-emitter coupled to a light source such that it is in communication with at least one predetermined site in the nervous system of a body; stimulating said at least one predetermined site in said nervous system of said body using said at least one light-emitter. In one aspect of the invention, the disorder being treating is Parkinson's disease, Alzheimer's disease, depression, or epilepsy. In one aspect of the invention, the above method further includes the step of regulating at least one parameter of said step of stimulating, said at least one parameter being selected from the group consisting of pulse width, pulse frequency, and pulse amplitude.

In one embodiment, the present invention relates to a method for treating a disorder in a patient comprising the steps of: surgically implanting a light-emitter into a brain of a patient wherein said light emitter is coupled to a light source and a signal generator operating said light source; and operating said signal generator to stimulate a predetermined treatment site in said brain.

### BRIEF DESCRIPTION OF THE DRAWINGS

FIG. 1 is a diagrammatic illustration of an light-emitter implanted in a brain according to a preferred embodiment of the present invention and a signal generator coupled to the light-emitter;

FIG. 2 is a diagrammatic illustration of a portion of the nervous system of the human body in which a preferred form of motion sensor, signal generator and light-emitter 25 have been implanted;

FIG. 3 is a schematic block diagram of a microprocessor and related circuitry used in a preferred embodiment of the invention; and

FIG. 4 is a flow chart illustrating a preferred form of a microprocessor program for generating stimulation pulses to be administered to the brain.

FIG. 5 is an illustration of implantation of a stimulator, and use within the vascular system.

FIGS. 6-8 present example target areas for stimulation, with related consequences.

FIG. 9 presents an example of placement of multiple light-emitters.

FIG. 10 presents additional examples of types of light sources.

FIG. 11 presents an exemplary nerve cuff as used in the methods herein.

### DETAILED DESCRIPTION OF THE INVENTION

### A. Definitions

Deep brain stimulation (DBS), as used herein refers to the stimulation of neural tissue using either conventional electrical stimulation methods, or using light stimulation methods as disclosed herein.

Light, as used herein, refers to optical energy or electromagnetic radiation. This optical energy may have any wavelength, including visible light as well of energy with longer and shorter wavelengths. This light may include laser light.

Light source, signal generator, as used herein refers to a source of light, electromagnetic radiation or optical energy. This source may be used to produce energy in order for this energy to be conveyed to a target tissue so that the target tissue is activated or inactivated by the optical energy. The light source may provide for pulsatile or modulated light to be produced. The light source may provide for short micropulses (eg 0.1-1000 picosecond) formed into trains within longer macropulses (eg 0.1-1000 microseconds) which in turn may be controlled in trains or other temporal patterns. The light source may be a laser light source or other light source. The light source may be controlled by a microprocessor, computer or computer program that determines the pattern, or signal, to be presented. Any among the light source, microprocessor, power supply, biocompatible protective casing, leads, and related hardware may be implanted within the subject.

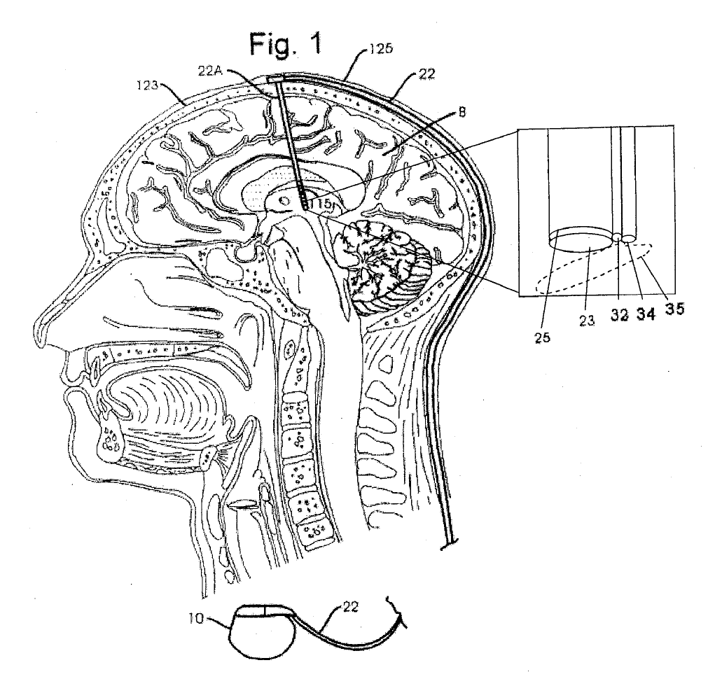

Light-emitter, as used herein, refers to a point from which electromagnetic radiation is given out, for example given out so that it strikes a target tissue. A light-emitter may be used as a stimulator of target tissue using light. A light-emitter may stimulate a target tissue using light or optical energy by propagating the light or optical energy into the target tissue. For example, a light-emitter may be the end of an optical fiber adjacent to a target tissue and through which light is conducted. An example is presented in FIG. 1, 25.

Light conductor, as used herein refers to a means to conduct light or optical energy from one location to another, including but not limited to an optical fiber.

Neuromoanatomical texts, as used herein refers to any of a variety of texts describing the structures of the brain that may be used as target tissues of this invention, including but not limited to Fundamental Neuroanatomy by Nauta and Feirtag, and in the Co-Planar Steriotaxic Atlas of the Human Brain by Jean Talairach and Pierre Tournoux, Magnetic Resonance Imaging of the Brain and Spine (2 Volume Set) by Scott W., Md. Atlas.

Neuromodulator or neuromodulatory substance, as used herein, refers to compounds which can alter activity or responsiveness in one or more localized regions of the brain. Examples of neuromodulators include, but are not limited to: opioids, neuropeptides, acetylcholine, dopamine, norepinephrine, serotonin and other biologic amines, and others. Many pharmacologic agents such as morphine, caffeine and prozac are exogenous mimics of these neuromodulatory substances.

Neuromodulatory centers, as used herein, refers to regions of the brain or nervous system that serve to regulate or alter responsiveness in other parts of the nervous system. Examples include regions that contain neurons that release neuromodulatory transmitters such as catecholamines, acetylcholine, other biologic amines, neuropeptides, serotonin, norepinephrine, dopamine, adrenaline. These centers and the actions produced through their modulation are described in neuroanatomy texts and The Biochemical Basis of Neuropharmacology, Cooper, Bloom and Roth. Examples include but are not limited to the nucleus raphe magnus, substantia nigra (pars compacta and reticulata), nucleus accumbens, periaqueductal gray, locus coeruleus, nucleus basalis, red nucleus, nucleus accumbens. These regions may serve as target tissues.

Optical fiber, as used herein, refers to a flexible substantially optically transparent fiber, usually made of glass or plastic, through which light can be transmitted by successive internal reflections. In addition, this invention discloses that other means for conveying light from a source to a precise spatial location may be used in place of an optical fiber.

Pharmacological treatment, as used herein, refers to the administration of any type of drug or medication.

Region of interest or ROI or volume of interest, as used herein, refers to a particular one or more voxels of the body, nervous system or brain of a subject. An ROI may occasionally be referred to as an area or volume of interest since the region of interest may be two dimensional (area) or three dimensional (volume). Frequently, it is an object of the methods of the present invention to monitor, control and/or alter brain activity in the region of interest. For example, the one or regions of interest of the brain associated with a given condition may be identified as the region of interest for that condition. In one variation, the regions of interest targeted by this invention are internal relative to a surface of the brain.

Reward centers or pleasure centers, as used herein, refers to areas of the brain which, when active, produce pleasurable or rewarding experiences or sensations. These include, but are not limited to certain limbic structures, the nucleus accumbens, locus coeruleus, septal nuclei, and others. These may also include areas that have been associated with addictive behaviors. These may serve as target tissues.

Single point, as used herein, refers to an individual geometric locus or small area of volume, such as a single small geometric volume from which a physiological measurement will be made, with the volume being 0.01, 1, 0.5, 1, 2, 3, 4, 5, 10, 15, 20, 30, 50, 100 mm in diameter. A device making a measurement from a single point is contrasted with a device making scanned measurements from an entire volume comprised of many single points.

Spatial array, as used herein, refers to a contiguous or non-contiguous set of single points, areas or volumes in space. The spatial array may be two dimensional in which case elements of the array are areas or three dimensional in which case elements of the array are volumes.

Spatial pattern, or spatial activity pattern, or vectorized spatial pattern, as used herein, refers to the activities of a set of single points forming a two dimensional or three dimensional spatial array. A vector comprising a value for each point in a three dimensional spatial array is one example of a spatial pattern, or a value for each point at each moment in time is another example of a spatial pattern.

Subject, as used herein, refers to a target whose activity is to be controlled in conjunction with performing the methods of the present invention. It is noted that the subject may be the person who has the condition being treated by the methods of the present invention. Subjects may also refer to animal subjects, or to target tissue taken from animals or humans.

Tissue or target tissue, as used herein, refers to biological tissues to which this invention may be applied. These tissues include, but are not limited to, excitable tissue, tissue in either the central nervous system, peripheral or cranial nerves, autonomic nervous tissue, smooth or striated muscle tissue, vascular tissue. These target tissues may be in humans or animals. These target tissues may be either in the in vivo setting (ie inside the subject) or may have been removed from the subject (eg for use in isolated tissue from the nervous system such as for study of a hippocampal or other slice preparation).

Referring to FIG. 1, a system or device 10 made in accordance with a preferred embodiment may be implanted below the skin of a patient. A lead 22A is positioned to stimulate a specific site in a brain (B). This stimulation may include stimulation of neuronal activity. Device 10 may take the form of a modified signal generator. Lead 22A may take the form of a light conductor, including an optical fiber, for stimulating the brain, and is coupled to device 10 by a light conductor 22.

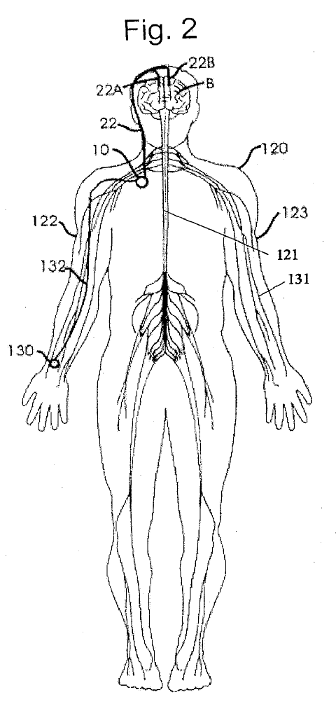

The distal end of lead 22A terminates in one or more stimulation light-emitters 25 generally designated a stimulator group 115 implanted into a portion of the nervous system, for example by conventional stereotactic surgical techniques. However, other numbers of light-emitters 25, such as 2, 3, 4, 5, 6-10, 10-20, 20-30, 30-50, 50-100, 100-200, 200-1000, 1000-5000, or 5000-10000 may be used for various applications or in some embodiments more than 2, 3, 4, 5, 6, 7, 8, 9, or 10 light-emitters can be used. Each of the light-emitters 25 is individually connected to device 10 through lead 22A and light conductor 22. Lead 22A may be surgically implanted through a hole in the skull 123 and light conductor 22 is implanted between the skull and the scalp 125 as shown in FIG. 1. In this regard, a lead may be an apparatus for conveying light, including one or more optical fibers. Light conductor 22 is joined to implanted device 10 in the manner shown. Referring to FIG. 2, device 10 is implanted in a human body 120 in the location shown. Body 120 includes arms 122 and 123. Alternatively, device 10 may be implanted in the abdomen, and emitters 25 may be placed adjacent to peripheral or cranial nerves 131, or in or near striated or smooth muscle.

Light conductor 22 may be divided into twin leads 22A and 22B that are implanted bilaterally as shown. Alternatively, lead 22B may be supplied with stimulating pulses from a separate conductor and signal generator. Leads 22A and 22B could be 1) two light-emitters 25 in two separate nuclei that potentiate each others effects or 2) nuclei with opposite effects with the stimulation being used to fine tune the response through opposing forces.

The light emitters 25 may be positioned by viewing tissue internal to the subject using a laparascopic or other camera connected to a viewing device 32 placed near to the light emitters. In addition, the light conductor 22, since it may conduct light in both directions, may be used alternately for viewing the internal structure of the subject, and subsequently for light stimulation. In addition, fluid, gas, substantially light transparent media 35, or other agents including drugs or pharmacological agents may be passed into the subject through a catheter 34 near to the light emitter, stimulator.

A sensor 130 is attached to or implanted into a portion of a patient's body suitable for detecting symptoms of a disorder being treated, such as a motor response or motor behavior. Sensor 130 is adapted to sense an attribute of the symptom to be controlled or an important related symptom. For motion disorders that result in abnormal movement of an arm, such as arm 122, sensor 130 may be a motion detector implanted in arm 122 as shown. For example, sensor 130 may sense three-dimensional or two-dimensional motion (linear rotational or joint motion), such as by an accelerometer. One such sensor suitable for use with the present invention is described in U.S. Pat. No. 5,293,879 (Vonk). Another suitable accelerometer is found in pacemakers manufactured by Medtronic, Inc. and described in patent application Ser. No. 08/399,072 filed Mar. 8, 1995, in the names of James Sikorski and Larry R. Larson and entitled “Package Integrated Accelerometer”. Sensor 130 also may be placed in device 10 in order to detect abnormal movement resulting from the motion disorder being treated.

Sensor 130 also may be capable of detecting gravity direction or motion relative to some object (e.g., a magnet) either implanted or fixed nearby. Sensor 130 also may take the form of a device capable of detecting force in muscles or at joints, or pressure.

Sensor 130 may detect muscle EMG in one, two or more muscles, or in reciprocal muscles at one joint. For such detection, sensor 130 may take the form of a recording electrode inserted into the muscle of interest.

Brain neurophysiological signals including single neuron recordings or EEG (e.g., motor cortex potentials recorded above the motor neurons controlling specific muscle groups) also may be detected by sensor 130.

Yet another form of sensor 130 would include a device capable of detecting nerve compound action potentials (e.g., either sensory afferent information from muscle or skin receptors or efferent motor potentials controlling a muscle of interest).

For certain types of patients, sensor 130 may take the form of device detecting the posture of the patient. Sensor 130 also may take the form of a device capable of detecting nerve cell or axon activity that is related to the pathways at the cause of the symptom, or that reflects sensations which are elicited by the symptom. Such a sensor may be located deep in the brain. For such detecting, sensor 130 may take the form of an electrode inserted into the brain. Signals that are received by the sensor may by amplified before transmission to circuitry contained within device 10.

Sensor 130 may take the form of a transducer consisting of an electrode with an ion selective coating applied which is capable of directly transducing the amount of a particular transmitter substance or its breakdown by-products found in the interstitial space of a region of the brain such as the ventral lateral thalamus. The level of the interstitial transmitter substance is an indicator of the relative activity of the brain region. An example of this type of transducer is described in the paper “Multichannel semiconductor-based electrodes for in vivo electrochemical and electrophysiological studies in rat CNS” by Craig G. van Home, Spencer Bement, Barry J. Hoffer, and Greg A. Gerhardt, published in Neuroscience Letters, 120 (1990) 249-252.

For tremor, the relative motion of a joint or limb or muscle EMG may be productively sensed. Sensing electrical activity of neurons in various locations of the motor circuitry also is helpful. Recording the electrical activity in the thalamus or cerebellum will reveal a characteristic oscillating electrical activity when tremor is present.

For Ballism, Hemiballism or tremor, sensor 130 may take the form of an accelerometer detecting relative motion of a joint and limb or muscle EMG.

For Dystonia, sensor 130 may take the form of a device for detecting relative motion of a joint or limb or muscle EMG.

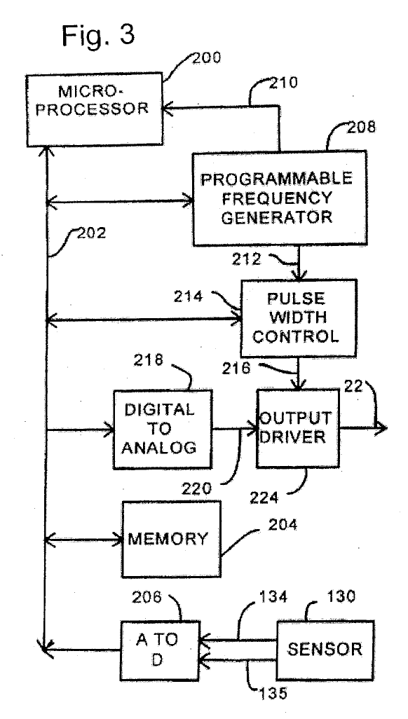

Referring to FIGS. 2 and 3, the output of sensor 130 is coupled by cable 132, comprising conductors 134 and 135, to the input of an analog to digital converter 206 within device 10. Alternatively, the output of an external sensor would communicate with the implanted pulse generator through a telemetry downlink.

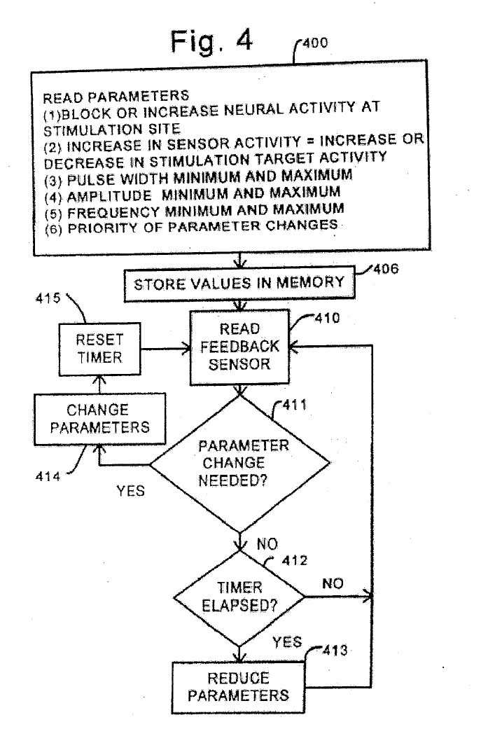

It may be desirable to reduce parameter values to the minimum level needed to establish the appropriate level activity in a target region. In FIG. 4, steps 410 through 415 constitute the method to adjust stimulation parameters using a feedback mechanism that detects a result of stimulation. When parameters are changed, a timer is reset in step 415. If there is no need to change any stimulus parameters before the timer has counted out, then it may be possible due to changes in activity to reduce the parameter values and still maintain appropriate levels of activity in the target, or in downstream processes effected by the target. At the end of the programmed time interval, device 10 tries reducing a parameter in step 413 to determine if control is maintained. If it is, the various parameter values will be ratcheted down until such time as the sensor values again indicate a need to increase them. While the algorithms in FIG. 4 follow the order of parameter selection indicated, other sequences may be programmed by the clinician.

Microprocessor 200 within device 10 can be programmed so that the desired stimulation can be delivered to the specific brain sites described. Alternatively, sensor 130 can be used with a closed loop feedback system in order to automatically determine the type of stimulation necessary to alleviate motor disorder symptoms as described in connection with FIG. 4.

Microprocessor 200 may execute an algorithm in order to provide stimulation with closed loop feedback control. At the time the stimulation device 10 is implanted, the clinician may program certain key parameters into the memory of the implanted device via telemetry. These parameters may be updated subsequently as needed. Step 400 in FIG. 4 indicates the process of first choosing whether the neural activity at the stimulation site is to be blocked or facilitated (step 400(1)) and whether the sensor location is one for which an increase in the neural activity at that location is equivalent to an increase in neural activity at the stimulation target or vice versa (step 400(2)). Next the clinician must program the range of values for pulse width (step 400(3)), amplitude (step 400(4)) and frequency (step 400(5)) which device 10 may use to optimize the therapy. The clinician may also choose the order in which the parameter changes are made (step 400(6)). Alternatively, the clinician may elect to use default values.

The algorithm for selecting parameters is different depending on whether the clinician has chosen to block the neural activity at the stimulation target or facilitate the neural activity. FIG. 4 details steps of the algorithm to make parameter changes.

The algorithm uses the clinician programmed indication of whether the neurons at the particular location of the stimulating light-emitter 25 are to be facilitated or blocked in order to reduce the neural activity in the subthalamic nucleus to decide which path of the parameter selection algorithm to follow.

It is desirable to reduce parameter values to the minimum level needed to establish the appropriate level of neuronal activity in the subthalamic nucleus. Superimposed on the algorithm just described is an additional algorithm to readjust all the parameter levels downward as far as possible. In FIG. 4, steps 410 through 415 constitute the method to do this. When parameters are changed, a timer is reset in step 415. If there is no need to change any stimulus parameters before the timer has counted out, then it may be possible due to changes in neuronal activity to reduce the parameter values and still maintain appropriate levels of neuronal activity in the target neurons. At the end of the programmed time interval, device 10 tries reducing a parameter in step 413 to determine if control is maintained. If it is, the various parameter values will be ratcheted down until such time as the sensor values again indicate a need to increase them. While the algorithms in FIG. 4 follow the order of parameter selection indicated, other sequences may be programmed by the clinician.

Appropriate stimulation pulses may be generated by device 10 based on the computer algorithm shown in FIG. 4 that read the output of converter 140 and makes the appropriate analysis.

For some types of conditions, a microprocessor and analog to digital converter will not be necessary. The output from sensor 130 can be filtered by an appropriate electronic filter in order to provide a control signal for device 10.

The type of stimulation administered by device 10 to the brain depends on the specific location at which the stimulator group 115 of leads 22A are surgically implanted. The appropriate stimulation for the portion of the basal ganglia or thalamus in which lead 22A terminates, together with the effect of the stimulation on that portion of the brain for hyperkinetic motion disorders is provided.

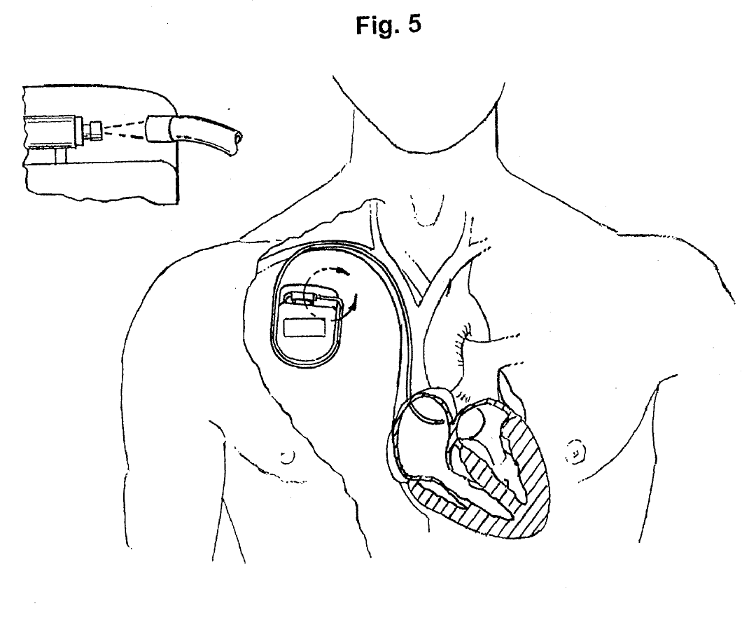

FIG. 5 shows an example of this invention that uses an implanted container 510 containing a battery 520 or other power source capable of driving a light source 530 that produces light and transmits it either directly to target tissue or through an optical fiber 540 to target tissue where it may optionally be focused through a lens onto a target tissue. In the example shown the target tissue is the heart 560, so the device is able to function as a pacemaker by activating atrial tissue. In addition, the optical fiber is passed through the lumen of a blood vessel.

Methods are provided for applications of stimulating target tissue wherein the source of stimulation is optical energy. This invention discloses applications of this basic principle, disclosed in U.S. Pat. No. 6,921,413. In certain embodiments of the method, a free electron laser is used as a source of optical energy. It is possible to use sources other than free electron lasers that are capable of generating the appropriate wavelengths, pulses, and energy levels.

### Implantable Light Sources

Using this invention the light source used for stimulation may be implantable as shown in FIG. 1. Implantable light sources 10 may be used as a replacement for electrical stimulators as they are used with electrical stimulation for long term chronic stimulation applications including, but not limited to, heart pacing, spinal cord stimulation, deep brain stimulation, cranial or peripheral nerve stimulation, vagal nerve stimulation. For an implantable light source a battery and light source such as a laser may be placed inside a biocompatible protective container with the light source. The light source may be a diode laser. Methods for implantable manufacture of batteries and light sources may be provided, for example, as described in U.S. Pat. No. 6,925,328—MRI-Compatible Implantable Device. In the currently disclosed invention, unlike in U.S. Pat. No. 6,925,328, light may be used for direct stimulation of target tissue, rather than light being converted into an electrical signal which is then used to stimulate tissue.

### Implantable Leads

Leads 22 for use in this invention may comprise light conductors as shown in FIG. 1. Leads for use in this invention may be optical fibers. Leads for use in this invention may be fiber optic bundles. In electrical stimulation methods disclosed previously, leads are typically electrical wires. Here, it is disclosed that implantable electrical leads may be replaced by leads that convey light in order to directly stimulate target tissue with light. In one embodiment, fiber optic leads used in this invention may be implantable and biocompatible. In another embodiment, the leads may be percutaneous, connecting a light source or laser outside of the body with a stimulus location inside the body. Methods and apparatus for percutaneous passage of lead wires used in electrical stimulation may be used for percutaneous passage of light conductors.

### Implantable Light-Emitters

In one embodiment, light-emitters 25 used in this invention may be implantable and biocompatible as shown in FIG. 1. This may have advantages over electrical stimulation electrodes in that electrical stimulation may lead to tissue necrosis or electrolytic reactions not present with light stimulation. Light-emitters 25 may include either the bare end of a fiber optic, may include a biocompatible lens, and may include a biocompatible window 23 through which light is presented.

### Window

Stimulation may be applied through a window 23 that serves to form a seal as shown in FIG. 1. This may allow stimulation minimizing risk of infection. The window 23 may be composed of material that is substantially transparent to the stimulating energy. The window 23 may be coated so as to prevent adhesion of biological materials or other obstructions to the light path. This window 23 may be held in place through an appliance that secures it to the skin, to bone, or to connective tissue. This may allow for light stimulation to regions internal to a subject while maintaining intact bodily boundaries of the subject.

### Conducting Medium, Laparoscopy

In addition, methods may be used to provide a conducting medium between the light-emitter 25 and the target tissue, which may be passed through catheter 34 as shown in FIG. 1. This conducting medium may comprise water, a solution substantially transparent to the stimulating energy, or gas. In one embodiment, a method may be provided to convey a gas or transparent solution into the body in order to displace internal organs or fluids so that stimulation using light may take place unobstructed by other internal organs or fluids. For example, a catheter placed alongside the light conductor may be used to pass a transparent fluid into the body of the subject. In this way, the internal aspects of the body of the subject may be visualized through a light conductor, for example using a laparascope. In addition, if a substantially transparent fluid or gas 35 is passed into the body, this may be used to allow light to pass from the light emitter to the target tissue. This transparent fluid may displace less transparent organs or bodily fluids of the subject. Aspects of the invention provided here may be completed in conjunction with conventional laparoscopic methods, for example as discussed in Cuschieri A. “Laparoscopic surgery: current status, issues and future developments.” Surgeon. 2005 June 3(3):125-30, 132-3, 135-8. For example, light stimulation of target tissues may be performed using laparascopic placement of one or more light emitter, and visualization through light conductor 32.

### Nerve Inactivation (as Opposed to Stimulation)

The disclosed device may be used for target inactivation. For example, using high frequency pulses may be used to inactivate neural tissue. The frequency may be 100-1000 Hz, 1000-5000 Hz, 5-10 kHz, 10-20 kHz, 20-50 kHz, 50-500 kHz or greater. Through applying a long (>1 s) train of repetitive light pulses, a target tissue may become fatigued, habituated, or otherwise inactivated.

### Monitoring of Activation Induced Using Neuroimaging, Fmri, Real Time Fmri

Activation resultant from stimulation using this device may be used in combination with any method for measuring biological tissue activation. In one embodiment, this invention provides for a nerve conduction study to be made in a subject through stimulating a nerve using light, and recording the resultant activation using recording electrodes following methods common in the art but previously using electrical stimulation of the nerve. This method may be used for nerve conduction studies in humans. This method provides for nerve conduction studies using any of the peripheral or cranial nerves. In addition, this device may be used in conjunction with fMRI as a measure of neural activation, or real time fMRI.

### Intravascular Implantation

In one embodiment, a light-emitter and lead may be implanted intravascularly, as shown in FIG. 5. This implantation may use a direct adaptation of methods familiar to one skilled in the art for the intravascular implantation of wire leads, substituting or adding a fiber optic lead and light-emitter to a wire lead. In one embodiment, stimulation may be of target tissue inside the vascular system, such as heart muscle tissue or other vascular tissue. In another embodiment stimulation may take place across the vascular wall, using light to stimulate target tissue beyond the vascular wall. In another embodiment the vascular system may be used as a conduit to place a light-emitter and lead, which then exit the vascular system through a vascular wall in order to stimulate target tissue outside of the vascular system.

### Light Sources and Parameters

This invention may employ any form of radiant energy sufficient to stimulate activity in target tissues.

In one embodiment, a free electron laser and delivery optics may be used to generate and manipulate the light, or optical energy. The optical energy transport system may be maintained under rough vacuum. The optical energy may be focused on the target neural tissue using focusing lenses (for example Vi Convex Lenses, f=300 mm) to a spot size of for example 400 micrometers. Optical stimulation may be performed using laser pulses with energy in the range from 0.2 mJ to 5 mJ with a spot size of 300-600 micrometers (fluence values varied from 0.2 j/cm² to about 10 J/cm²). The minimum energy and therefore fluence required to stimulate a frog nerve preparation as described in U.S. Pat. No. 6,921,413 may be minimum (0.6 J/cm.²) between 4 and 4.5 micrometers. The laser pulses may be focused onto the sciatic nerve using Biconvex Lenses. The laser pulse energy may be varied using a polarizer.

The FEL may offer the flexibility of providing various wavelengths in the infrared spectrum for use with the method provided herein. Other sources may be used to generate the necessary wavelength. In addition to any source that can generate wavelengths in the infrared portion of the spectrum, sources may include LED and LCD. FEL additionally may provide micropulses, each about 1 picosecond in duration and having a repetition rate of about 3 GHz. The envelope of this pulse train may forms macropulse that is about 3-6 microseconds and may be delivered at a rate up to 30 Hz or higher. As mentioned above, optical stimulation of the peripheral nerves may employ pulse energies ranging from 0.2 mJ to 5 mJ in a spot size of around 500 micrometers.

Stimulation studies can also be performed using other sources such as a YAG laser for wavelengths in the UV, visible and infrared. Additionally, if it is desired to use a wavelength around 4 micrometers, then a lead-salt laser, or an optical parametric oscillator (or amplifier) may be used.

Various light wavelengths from 2 micrometers to 6.45 micrometers may be used to stimulate neural tissue. FEL wavelength of 6.45 micrometers may be effective, possibly due to the amid II vibrational band of protein (Edwards, et al., Nature, 371(6496): 416-419, 1994). While using the wavelength of 6.45 micrometers, nerve stimulation may occur at a pulse energy of 4.5-5.0 mJ/pulse, with a spot size measured of close to 0.5 mm.

Optical energy without a wavelength around the water absorption peak, at 2.94 micrometers, may be used for optical stimulation. In addition, using wavelengths of 3.1 micrometers and 3.3 micrometers may provide a nerve response however, these wavelengths may have a greater potential for causing damage to the neural tissues.

By using wavelengths in the range from 3.8 micrometers to 5.5 micrometers, a valley for the water absorption, the effects of photo-ablation may be minimized. Wavelengths around 4 micrometers may be more efficient in eliciting nerve response compared to other tested wavelengths.

Since FEL emits continuous laser pulses, an electromechanical shutter may be used to select a single pulse from the pulse train. Melles Griot (Irvin, Calif.) electronic shutter is used for gating laser pulses to obtain a single pulse from the pulse train. The shutter controller may be triggered using the trigger pulse from the laser.

Optical energy may be focused in a spatial area in the range of 1-5, 5-10, 10-20, 20-50, 50-100, 100-200, 200-500, 500-1000, 1000-5000, 5000-10000, 10000-100000 micrometers. Also, the target neural tissue may receive the optical energy for an amount of time necessary to provide a stimulation effect. The optical source may be pulsed. In one embodiment each energy pulse may be in a range of from 1 picosecond to 10 picoseconds micropulse and from 1 to 10 microsecond macropulse. The wavelength used is a wavelength that may approximately correspond to a valley for water absorption of a neural tissue. Such valleys of water absorption may be in the wavelength ranges of 0.9 micrometers to 2.7 micrometers and 3.8 micrometers to 5.5 micrometers. Additionally, the wavelength used may be approximately 4.5 micrometers, approximately 2.2 micrometers, or approximately 1.23 micrometers. In other embodiments, the wavelength may be 4.4 micrometers, the energy output may be 1.5 mJ, the optical energy may occur in an area of 600 micrometers. In other embodiments micropulses may be in the range of: 0.1-1, 1-10, 10-100, 100-1,000, 1,000-10,000, 10,000-100,000 picoseconds or less than 1, 0.5, 0.1, 0.05, 0.01, 0.005, 0.001, or 0.0005 microseconds. Macropulses may be in the range of: 1-10, 10-100, 100-1,000, 1,000-10,000, 10,000-100,000 microseconds or greater than 1, 5, 10, 50, 100, 500, 10,000, 50,000 or 100,000 microseconds.

In addition to these parameters, this invention provides for the possibility of adjusting the light source, light delivery method, wavelength, pulse width, pulse amplitude, and pulse duration of stimulating light to produce activity, and then using the selected parameters for further stimulation as provided here. In addition, as further research into light stimulation produces optimized light sources, light delivery methods and parameter sets, these may be used in conjunction with this invention.

### Hardware

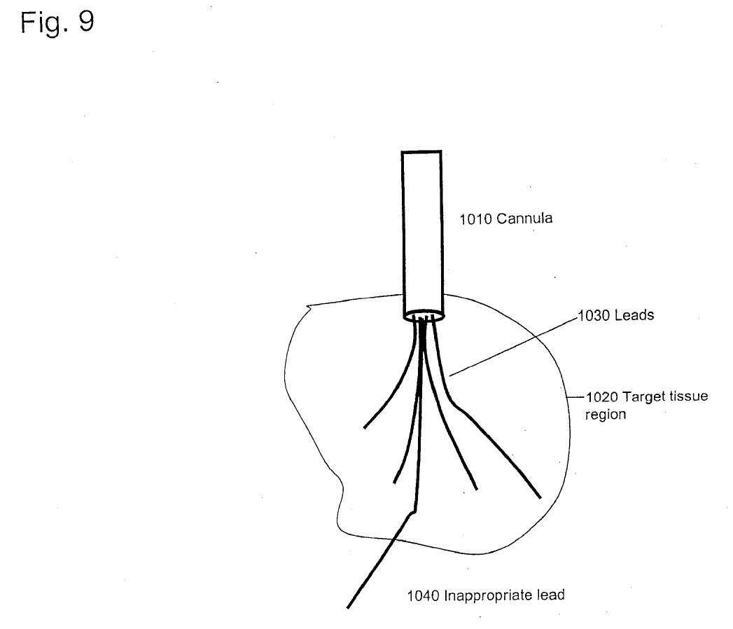

A group of multiple light emitters 115 may be individually controlled to pass through separate light conductors 22, with each light conductor positioned at a different target location. In this way, by differentially applying light through each light conductor, spatial patterns of activation may be presented that activate different combinations of locations of neural tissue. In addition, this multi-channel stimulation configuration, as shown in FIG. 1, may be computer controlled using a computer within device 10 so that spatial patterns may be created. This may be completed using optical switching technologies within device 10. The computer controller may select which light conductors receive light pulses, and the exact times, durations, and intensities of stimulation. In this way, complex spatio-temporal patterns of stimulation may be produced on the target neural tissue. This may also be seen in FIG. 9. This allows different neural elements to be stimulated in arbitrary patterns in space and time.

The following components may be used in combination with this invention: free space beam, grin lens, tunable laser, solid state laser tunable by interferometer changes the length of the cavity, chemical lasers.

### Stimulation Parameters

In electrodes, ‘electrical targeting’ can be used to control neural activation by controlling the spread of the electric field and by selectively activating neural elements (Kuncell, et. al. 2004). Similarly, using light stimulation it is possible to select the stimulation parameters used to specify what neural elements will be stimulated.

Along with accurately placed electrodes, successful DBS depends on properly set stimulus parameters, including pulse width, frequency, and amplitude (Su et al., 2003). Typical DBS parameter settings using electrical stimulation may include voltage, pulse width, and frequency range from 1-3.5 V, 60-210 ms, and from 130-185 Hz (Moro et al., 2002; O'Suilleabhain et al., 2003; Rizzone et al., 2001; Volkmann et al., 2002). In a study comparing the efficacy of GPi and STN DBS, the final mean stimulus parameter settings used to treat PD symptoms were 3 V, 82 ms, and 152 Hz for STN DBS, and 3.2 V, 125 ms, and 162 Hz for GPi DBS (Obeso et al., 2001). Similarly, pulse width, frequency, and amplitude must be accurately selected for light stimulation of neural tissue. Light stimulation durations and frequencies may be based upon those found successful in electrical stimulation. Light stimulus parameters may be used to control selectively which neural elements in the surrounding tissue are excited. The stimulus parameters may also control the spatial extent of neural elements which are excited.

Stimulation frequencies using light stimulation may be substantially similar to those using electrical stimulation. Pulse widths may be substantially shorter.

In order to select the appropriate pulse width and amplitude, each of these two parameters may be varied independently while the stimulation response is measured in order to determine the optimal combination of pulse width and amplitude that just produces a change in activity in the target tissue while depositing a minimum amount of energy, or eliciting minimum tissue damage. Then, this pulse width and amplitude combination may be used at a repetition frequency appropriate to stimulate or inhibit the activity of the target region. The optimal combination may best reduce symptoms, minimize side effects, and minimize power consumption. Low power consumption may increase battery life and decrease the risk of tissue damage. Short pulse widths minimize charge in electrical stimulation, as explained by the charge-duration relationship. Reduced charge minimizes the probability of inducing tissue damage. Theoretical studies indicate that short pulse durations increase the threshold difference between activation of different diameter nerve fibers (Gonnan and Mortimer, 1983) and between activation of nerve fibers lying at different distances from the electrode (Grill and Mortimer, 1995). Empirically, short pulse widths may be found to increase the dynamic range between clinical benefit and adverse side effects, also referred to as the therapeutic window. Rizzone et al. (2001) determined the pulse width/stimulus intensity relationships for reduction of wrist rigidity in patients with PD and for onset of side effects. As the pulse width is decreased, the stimulus intensity required to elicit a clinically significant improvement may increase, which may be explained by the strength-duration relationship. The stimulus intensity causing side effects also may increase as the pulse width decreases, but the difference between the two amplitudes, the size of the therapeutic window, may increase as the pulse width decreases. Cumulatively, these results suggest that DBS devices may be programmed with the shortest possible pulse duration, and that future generation stimulators may include lower ranges of pulse widths. Similarly, the most appropriate pulse width for light stimulation may be derived. Shorter pulses may be selected to decrease tissue damage. High frequency stimulation may require more power, and therefore decreases battery life. DBS may be effective for reduction of tremor, akinesia, and rigidity at frequencies greater than 50 Hz but larger stimulus amplitudes may be required at low frequencies (Benabid et al., 1991; Limousin et al., 1995). Tremor suppression at the lowest current may occur between 150 and 1000 Hz, and the lowest stimulus intensity required may be about 2 mA (Benabid et al., 1991). Above 1000 Hz, the efficiency of tremor suppression may decrease, presumably as a result of neural refractoriness. The clinical effect of STN stimulation on akinesia and rigidity may be studied with similar results (Limousin et al., 1995). The stimulus amplitude required to activate neural elements depends on the spatial relationship between the electrode or light-emitter and the nerve fiber (McNeal, 1976). As the distance between the active contact and the neural element is increased, the stimulus amplitude required to stimulate neural elements increases non-linearly. DBS studies have shown that the clinical benefits saturate above a certain value.

### Stimulation Patterns

In some embodiments it is preferable to use spatial patterns of stimulation emanating from multiple stimulation sources. In one example, multiple stimulation contacts are inserted into neural tissue so that each stimulation contact is in a different location. These locations may span different neural elements, such as slightly different locations in a brain nucleus, cortical area, or part of the spinal cord, peripheral nerve or muscle tissue. Then, the amount and timing of stimulation from each of the stimulation contracts may be individually adjusted so that the greatest stimulation of the tissue is achieved. The amount and timing of stimulation from each of the stimulation contracts may also be individually adjusted to minimize tissue damage with similar resultant stimulation or inhibition. The amount and timing of stimulation from each of the stimulation contracts may also be individually adjusted to maximize the long-term effectiveness of stimulation over repeated stimuli (eg decreasing habituation). In addition, spatiotemporal patterns of stimulation to different sites may be controlled so that different spatial locations are stimulated at different times. This may be important in producing precisely timed resultant patterns of stimulation in the target tissue. For example, in muscle tissue individual muscles may be stimulated a different times in a precise spatiotemporal pattern in order to produce a coordinated movement. Similarly, different neural elements may be stimulated in a spatiotemporal pattern to achieve or mimic desired patterns of neural activation or inhibition. These spatiotemporal patterns may be adjusted by adjusting the timing or intensity of stimulation at each component stimulation site in order to optimize a desired response, such as a sensation, movement, or decrease in symptoms in the subject. This may also be used to produce precisely controlled stimulation patterns in experimental preparations that can be used to investigate the results of these patterns.

### Stimulation Locations

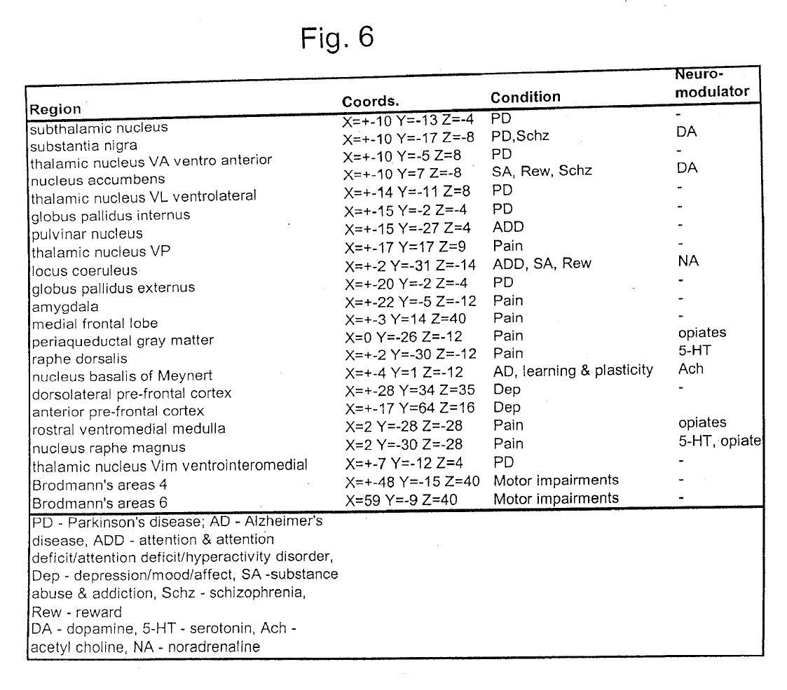

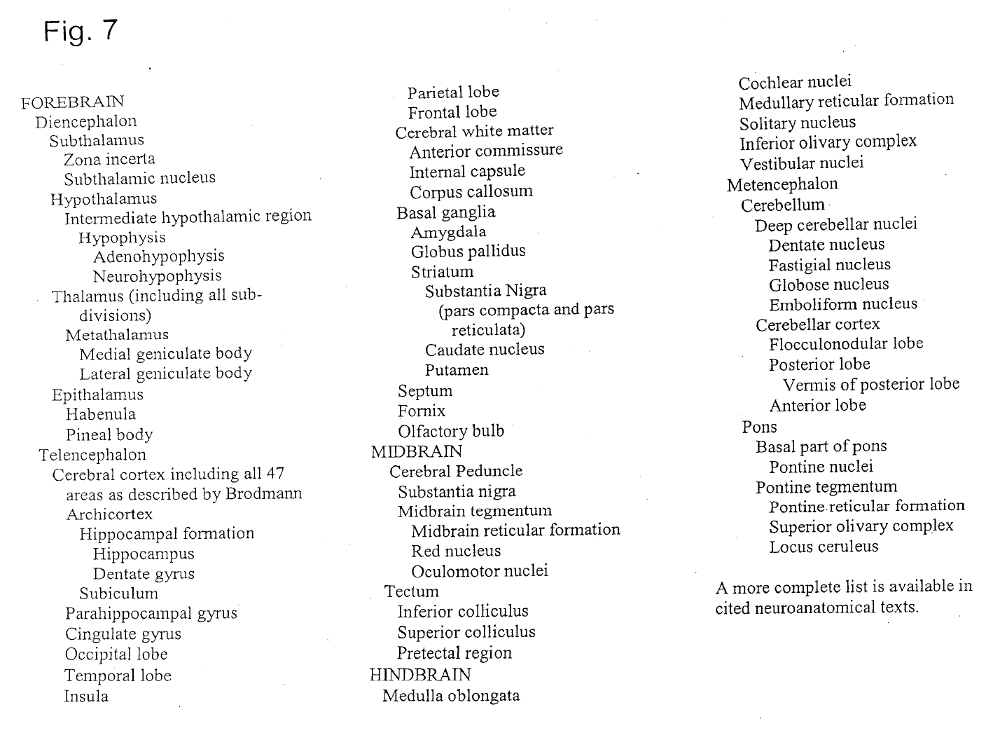

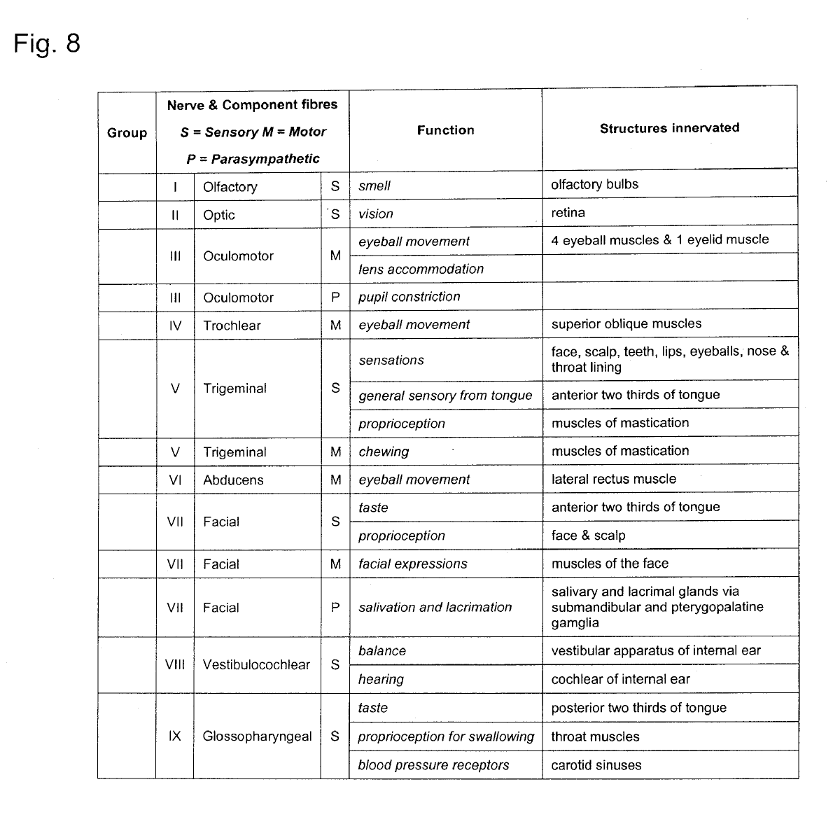

In addition, the invention disclosed here may be used for the stimulation of the following neural structures, through the acute or chronic placement of a stimulator inside or adjacent the these structures. The light may be conducted to the location through a light conductor, which may in turn be delivered through a canula, tube, or other method of delivery. Structures which may be stimulated include, but are not limited to: Spinal Cord, Subdural, Dorsal horn, Ventral horn, Nerves, Cranial nerves #1-12, Peripheral nerves, Nerve roots. Stimulation locations include, but are not limited to those depicted in FIG. 6-8. Additional tissue targets and stimulation locations may be found in neuroanatomical texts.

### Spinal Cord Stimulation

Using this invention the spinal cord 121 may be stimulated, replacing or supplementing the results of electrical spinal cord stimulation. This may be used in indications where electrical stimulation of the spinal cord is indicated, such as in the treatment of chronic pain. Light stimulation may be provided directly against neural tissue. If a suitable wavelength and stimulation parameters are available, stimulation may be made through intervening tissue.

Spinal cord stimulation is a method for stimulating or inhibiting neural elements of the spinal cord, and thereby impacting their physiological functions. Using this invention spinal cord stimulation may be applied using light rather than or in addition to the prior approach using electrical stimulation.

Some clinical indications for spinal cord stimulation are:

Vascular pain: refractory angina and
peripheral vascular diseases (PVD).
Rachidian pain: failed back surgery syndrome
(FBSS), degenerative low back-leg
pain (LBLP), spinal stenosis, nerve-root avulsion,
incomplete spine lesion.
Chronic regional pain syndromes (CRPS)
type I and II.
Neuropathic perineal pain:
Urological diseases: interstitial cystitis, urge-incontinence.

### Deep Brain Stimulation

This invention provides for deep brain stimulation using light as shown in FIG. 1. The target of deep brain stimulation may be a brain region internal to the brain B. The light for deep brain stimulation may be conveyed to the target tissue using a light conductor 22. Deep brain stimulation may use an implantable device 10.

Successful treatment with DBS depends on accurately placed electrodes or light-emitters. Anatomical targeting involves determining where to place the stimulator and where to direct the electric current, based on which neural elements, cells or fibers, are targeted for excitation. The STN (sub thalamic nucleus) is a common target for the treatment of Parkinson's disease (PD), and targeting the STN for treatment of PD results in clinically effective outcomes (Krause et al., 2001; Kumar et al., 1998; Limousin et al., 1995). The STN is a small nucleus, surrounded by several large fiber tracts, including the zona incerta (ZI) and the Fields of Forel (FF).

Light-emitters placed in the STN and the surrounding fiber tracts may elicit similar clinical improvements (Hamel et al., 2003; Saint-Cyr et al., 2002; Voges et al., 2002; Yelnik et al., 2003). However, Saint-Cyr et al. (2002) found that the best efficacy and fewest adverse side effects may occur most commonly when electrode contacts are located in the anterior-dorsal STN and/or in the FF/ZI dorsally adjacent to it. Hamel et al. (2003) found that active contacts located at the border between the STN and the area containing the ZI, FF, and STN projections may require the least voltage to alleviate rigidity. Voges et al. (2002) found that, for a similar clinical improvement, contacts located in the fiber tracts may require less stimulation power (where Power ¼ (Amplitude £ Pulse Width £ Frequency)2/Impedance) than those located in the STN. Similarly, subthalamotomies that extended beyond the STN into the FF/ZI may be more effective in the treatment of PD patients than lesions that not extending beyond the STN (Patel et al., 2003). Fiber tracts around the STN, the activity of which may be influenced by STN DBS, may play a role in mediating the motor effects of STN DBS (Voges et al., 2002). According to previous animal studies, neural elements up to 5 mm from the cathode may be affected by stimulation using stimulus amplitudes (3 mA) that may be used in DBS (Ranck, 1975).

Therefore, stimulation in the STN may spread to the surrounding fiber tracts (Voges et al., 2002). The globus pallidus (GP), or pallidum, is another target for DBS treatment of PD. Stimulation of the GP, which is comprised of the GPi and the external globus pallidus (GPe), may result in different clinical effects with electrodes placed in the GPi or the Gpe (Bejjani et al., 1997; Krack et al., 1998; Yelnik et al., 2000). DBS applied to the GPe or the area between the putamen and GP (13 out 14 contacts) may result in improved upper limb akinesia, whereas stimulation applied to the GPi (11 out of 12 contacts) may result in worsened upper limb akinesia. Contacts located at the border of the GPe and GPi may have mixed clinical effects. Rigidity may be improved for contacts located throughout the GP, including the area between putamen and GP, in the GPe, in the area between the GPe and GPi, and in the GPi.

The invention disclosed here may be used to provide precisely located stimulation of deep brain structures. Light may be applied to deep brain structures, such as brain nuclei, so that the desired target regions are stimulated by the light while other or surrounding regions are stimulated substantially less or not at all.

### Stimulation of Brain or Cortical Tissue

In one embodiment this invention may be used to stimulate tissue of the cerebral cortex within the brain B, shown in FIG. 1. Many structures of the cerebral cortex are ‘mapped’ so that different points on the cortical surface correspond to different features, such as points in visual space, points on the body surface, or sound frequencies. Therefore, this invention provides for the creation of patterns of activity in cortical tissue through stimulation at selected intensity levels at one or more points within cortical tissue. By applying a stimulus pattern, this pattern may be used to mimic information represented in electrical activity in brain tissue. For example, in the primary visual cortex each point in the brain corresponds to a location in visual space, and stimulation of each point may produce the percept of an image at that point in visual space. Therefore, by stimulating a pattern of points in the visual cortex of a subject, it is possible to mimic the representation by the subject's cortex of an image that is viewed by a subject.

This method also provides for the mapping of cortical or other brain tissue. A light stimulus may be presented to a target location, and the result may be observed, for example by determining whether the subject has a resultant perception, movement, or perturbation of a cognitive function such as language. Through repeating this procedure, it is possible to form a map of the functions of different brain areas. This map may be used to avoid important brain areas during invasive surgery.

This invention provides that stimulation of target tissue, such as brain tissue, may take place through multiple light-emitters being placed such as to illuminate multiple points of target tissue.

In addition, this invention provides that stimulation of target tissue may take place through moving the light spot produced by a single light source so that the light spot is scanned across the tissue. The intensity of stimulation at each point in the tissue may be adjusted by rapidly scanning the light spot to a pattern of positions in the target tissue, and selecting the intensity, pulse width, or other parameters of the light so that each position may receive a different stimulation intensity. Stimulation of brain tissue may take place during exposure of the brain tissue, for example during surgery. This may be used to determine the function of the target tissue being stimulated, for example by observing the effects of stimulation, or having the subject report the effects of stimulation. Stimulation of brain tissue may also take place using implanted stimulation apparatus. This therefore provides for long term or chronic stimulation of brain or cortical tissue using light.

### Stimulation of Brain Nuclei

In another embodiment, this invention may be used to stimulate one or more brain nuclei. This may take place through placement of one or more light emitters within or adjacent to the brain nucleus that will be stimulated.

### Placement of Multiple Leads Via Cannula

In another embodiment, multiple light stimulation leads and light-emitters may be positioned into different spatial locations within the target tissue. An example is presented in FIG. 9. In this example, a guide cannula 1010, for example an 18 gauge catheter, may be inserted into target tissue region 1020, for example into a brain nucleus such as the STN. Multiple light stimulation leads 1030 may be passed into the target tissue. These multiple leads may be passed into the target tissue through a cannula 1010 so that they enter the target tissue. It may be desirable for the leads to enter different locations in the target tissue. The tensile properties of the leads, or of supporting elements attached to them, may be designed to produce the effect of leads entering the target structure in particular directions or achieving a desired final shape so that their tip reaches a targeted final location. For example, if each lead is designed to bend at a different circular radius and exit the cannula at a different angle, this will produce the effect of the tip of each lead, and the light-emitter, reaching a different final target location with the target locations surrounding the end of the cannula. This provides for the possibility that each lead will stimulate a different location in the target tissue.

The level of stimulation through multiple leads may be individually controlled so as to provide spatial control over the areas in target tissue that are stimulated. The level of stimulation through the light-emitter at the end of each lead may be individually controlled. The result of stimulation of each individual light-emitter may be assessed in terms of the results of its stimulation. For example, in stimulating tissue adjacent to a lead placed into the STN of a Parkinson's patient, the results on a patient symptom such as tremor may be evaluated when different stimulation parameters such as stimulus intensity or timing pattern are used with that lead. Then, once a level of stimulation suitable to produce a desirable effect on patient symptoms has been determined, this level or a fraction of this level may be used for future stimulation through this lead in combination with stimulation through other leads using parameters determined in a similar fashion. In addition, some inappropriate leads 1040 may enter areas that are not within the target tissue region. Through determining that stimulation through a lead does not produce desired results, such as a decrease in tremor, or produces undesired results, such as patient muscle twitches, it may be possible to determine that a lead is not in a desired target location and is therefore an inappropriate lead. Stimulation through inappropriate leads may thereafter be avoided. In this way, the stimulation of inappropriate leads that are not in the target region may be minimized. This method allows for the stimulation using light of a spatial region within the target tissue. This method also allows for the avoidance of stimulation of undesirable regions.

### Using a Light Conductor to Guide Placement

Light conductors 32, optical fibers or fiber optic bundles may be used to guide the placement of stimulating light-emitters according to this invention as shown in FIG. 1. If an optical fiber's distal end is entered into the body of a subject, and the proximal end is connected to a light monitor or camera, then it is possible to use the optical fibers to make observations near the location of the distal end of the optical fibers within the subject's body. Methods for viewing target tissues through optical fibers have been well-developed in the field of laparoscopy and are familiar to one skilled in the art. Methods of placement using catheters, guide wires or methods of visualization have been well described in the literature. These methods may be used in the placement of light-emitters disclosed in this invention. In this way, it may be possible to visualize the location of placement of a stimulating element such as a light-emitter being implanted into target tissue. Light for illumination of the target tissue may be provided through one or more of the optical fibers by conveying light from the proximal end to the distal end, or light may be provided through a different source.

### Experimental Stimulation of Isolated Neural Tissue

Tissue from a subject may be removed from the subject and stimulated using this invention. For example, a hippocampal slice preparation may be removed from a rat or other experimental animal, and placed in an experimental apparatus for maintaining its physiological function according to methods familiar in the art, for example as described in Schmitz, D., Frerking, M. and Nicoll, R. A.: Synaptic activation of presynaptic kainate receptors on hippocampal mossy fiber synapses. Neuron 27:327-338 (2000). This may then be used as the target tissue for stimulation using the methods disclosed here. Whereas isolated neural tissue is often stimulated with one or more stimulating electrodes, this invention provides for the stimulation of this tissue by one or more light-emitters. Since a light-emitter does not generate a stimulus artifact, superior electrophysiological recording of resultant neural activity may be achieved. In addition, the present invention provides for very precise spatial and temporal patterns to be generated, and the resulting physiological changes may be measured. For example, light stimuli may be applied to a large number of locations in a section of isolated neural tissue, such as a hippocampal slice, and the resultant neural activity may be measured. The isolated neural tissue used as a target may also include cultured neurons, organotypic cultures, and other forms of maintained neural tissue. The stimulation used may be scanned to multiple points on the neural tissue to provide a precise spatial pattern of stimulation. For example, a single neuron may be stimulated through controlling stimulating light falling on different points on the neuron's axons, dendrites, cell body, or on fibers incident on the neuron.

### Mri Compatibility

This invention may be used to provide MRI compatible stimulation of tissue. This invention does not require implantation of metal lead wires that may not be MRI compatible. Light may be conveyed to the light-emitter by MRI compatible optical fibers. In addition, the placement of light-emitters may be guided by MRI, CT, or fluoroscopy. In addition, by placing an MRI receive coil adjacent to an implanted light-emitter or a guide wire used in it's placement, and making MRI measurement from this MRI receive coil using an MRI scanner, it is possible to precisely visualize the location of implantation and surrounding tissue.

### Retinal Stimulation for Site Impairment

The methods described herein may be used to achieve stimulation of the visual system. This stimulation may take place at the level of the retina, or at higher levels of the visual system including the optic nerve, optic tract, optic chiasm, lateral geniculate nucleus, primary visual cortex, or higher visual cortical areas. This may be used to achieve prosthetic effect, for example for the partial restoration of site in a visually impaired person. For example, by using pulsed laser excitation one may activate neural tissue of the retina or optic nerve. This may be used in patients to produce vision restoration. Stimulation of the retina may be applied through the pupil of the eye by formation of an image on the retina. In cases where electrical stimulation of the visual system has been employed, optical stimulation using this invention may be employed instead. For example, methods are disclosed in Sachs and Gabel, ‘Retinal replacement—the development of microelectronic retinal prostheses—experience with subretinal implants’, 2004 for visual system prostheses. Rather than using electrical stimulation as described, optical stimulation using this invention may be applied to the corresponding points in the visual system. In one embodiment, a retinal prosthesis may be constructed using an array of multiple light-emitters, each placed against a position on the retina. The stimulation may be computer controlled, so that the exact position, time, and intensity of stimulation on the retina may be precisely controlled. In addition, a sequence of stimulation may be employed so that different locations on the retina (or other neural structure) are stimulated in rapid succession.

Use in Virtual Retinal Display

In addition, stimulation may be accomplished by scanning a laser illumination spot to different points on the retina in rapid succession. Through modulating the laser pulse intensity at each location when it is reached, a different level of stimulation may be achieved at each location. This may be used to achieve visual activation, for example in macular degeneration, glaucoma, or other vision impairments. This process of scanning and modulation may also be used with a pulsed laser. The target point of each pulse may be scanned to different points on the retina, for example in a rectangular grid, and a laser pulse may be applied at each point that corresponds to the intensity level of the image being applied at that point. This is analogous to the analog process used in scanning a beam to different locations on a CRT monitor in order to form an image. In this case, however, stimulation of the retina or a visual system structure may take place through direct action of light on target tissue, rather than through the process of phototransduction through photopigments in photoreceptor cells. In this way, direct stimulation of target tissue may be possible in cases where phototransduction is not normally operational. The image formed may be captured in real time using video or other equipment. This provides for video, or a rapid succession of images, to be applied to the target tissue as a spatial and temporal pattern of light intensity or light pulses applied to each point on the target tissue. In addition, in order to properly focus the laser pulse on the retina, a lens may be used, or a contact lens may be placed upon the eye. The conventional methods of virtual retinal display are described in, for example, Viirre E, Pryor H, Nagata S, Furness T A “The virtual retinal display: a new technology for virtual reality and augmented vision in medicine”, Stud Health Technol Inform. 1998; 50:252-7 and in “Virtual Retinal Display (VRD) Technology”, presented in www.cs.nps.navy.mil/people/faculty/capps/4473/projects/fiambolis/vrd/vrd_full.html. In these methods, the current invention discloses that laser light presented through a virtual retinal display may be used for direct neuronal stimulation, in addition to phototransduction.

### Disease Conditions and Indications

The disclosed invention may be used in the treatment of a variety of diseases involving the nervous system. In particular, in any case where electrical stimulation of the nervous system has been applied, optical stimulation may be applied instead. Optical stimulation may also be applied in addition. For example, deep brain stimulation of the subthalamic nucleus for Parkinson's disease may be substituted using optical stimulation in a substantially similar location within the brain. This may be accomplished by passing a fiber optic or fiber optic bundle into the corresponding location, and applying pulsed stimulation of the neural tissue. This stimulation may take place through a stimulation window 23.

Any of a variety of conditions may be treated using this invention as a replacement for electrical or magnetic stimulation. In particular, in a variety of conditions where electrical stimulation of neural tissue using a conventional electrode has been described, such as those described in appendix material cited here, optical stimulation of neural tissue may be used as a replacement as disclosed here. These include, but are not limited to those depicted in FIG. 6-8.

### Measurement of Activity

This invention may be used in conjunction with a variety of methods for measuring physiological activity from a subject. Examples of measurement technologies include, but are not limited to, EEG, single neuron recording, EMG, ECG, nerve potential recording, functional magnetic resonance imaging (fMRI), PET, SPECT, magnetic resonance angiography (MRA), diffusion tensor imaging (DTI), ultrasound and doppler shift ultrasound. It is anticipated that future technologies may be developed that also allow for the measurement of activity from localized regions, preferably in substantially real time. Once developed, these technologies may also be used with the current invention. These measurement techniques may also be used in combination, and in combination with other measurement techniques such as EEG, EKG, neuronal recording, local field potential recording, ultrasound, oximetry, peripheral pulsoximetry, near infrared spectroscopy, blood pressure recording, impedence measurements, measurements of central or peripheral reflexes, measurements of blood gases or chemical composition, measurements of temperature, measurements of emitted radiation, measurements of absorbed radiation, spectrophotometric measurements, measurements of central and peripheral reflexes, and anatomical methods including X-Ray/CT, ultrasound and others.

Any localized region within the brain, nervous system, or other parts of the body that is measured using physiological monitoring equipment as described (or other physiological monitoring equipment that may be devised) may be used as the target of this method. For example, if measurement equipment is used for the monitoring of activity in a portion of the peripheral nervous system, such as a peripheral ganglion, then subjects may be stimulated for the regulation of activity of that peripheral ganglion. In addition, the monitored point may be downstream or affected by the region being stimulated. For example, if a nerve is stimulated, measurements may be made at a distal muscle or effector organ that is innervated or activated by the nerve.

### Example Experimental Preparation

One example of the use of this invention is to use the rat sciatic nerve for frog isolated nerve preparation, as described in U.S. Pat. No. 6,921,413, for the target tissue which may serve as an example of its application. One of ordinary skill in the art understands the differences in the surgical procedure necessary to expose the Rat sciatic nerve. Regarding the stimulation of the Rat sciatic nerve, a wavelength of 4.4 micrometers, and energy of 4.7 mJ, a spot size of 619 micrometers, and a pulse frequency of 2 Hz using the FEL may be used. Optical stimulation may also use an energy of 39 mJ, 1.78 mJ, and 2.39 mJ.

The present invention described herein provides methods of stimulating target tissue with optical energy. In other embodiments, the present invention provides a method of stimulating neural tissue by providing a source capable of generating an optical energy having a wavelength in a range of from 3 micrometers to 6 micrometers at an energy output in a range from 200 microjoules to 5 millijoules, providing a target neural tissue, and focusing the optical energy on the target neural tissue so that the target neural tissue propagates an electrical impulse. A source of optical energy that may be used is a free electron laser. Examples of target neural tissues include a mammalian nerve, a human nerve, a sciatic nerve from a leopard frog in a model system.

The response of sciatic nerve to the optical energy stimulation may be sensed using stainless steel needle electrodes that are placed under the sciatic nerve for compound nerve action potential recording. Additionally, the electrical response from the sciatic nerve may be monitored by recording electrodes placed in the nerve downstream and innervated hamstring muscle. If the sciatic nerve conducts an electrical impulse, a tiny electrical signal may be detected from the nerve and a much larger electrical signal can be detected from the muscle. The signals may be recorded using the MP100 system from Biopac Systems (Santa Barbara, Calif.) which is combined electrical stimulation and recording unit. For comparison purposes, the nerve may be electrically stimulated using S44 Grass electrical stimulator from Grass Instruments, Quincy, Mass. At varying fluences, individual nerve fiber diameters and excitation thresholds may vary by small increments.

The present invention, herein described, provides a method of stimulating a nerve fiber by providing an optical source capable of generating an optical energy having a wavelength in a range of from 1 micrometers to 8 micrometers at an energy output in a range of 150 microjoules to 5 millijoules, providing the target nerve fiber, and focusing the optical energy on the target nerve fiber so that the target nerve fiber is stimulated. The target nerve fiber can be a mammalian nerve fiber, a human nerve fiber, or a leopard frog sciatic nerve fiber. During focusing, the target nerve fiber receives the optical energy for an amount of time necessary to provide a stimulation effect. The optical source can be pulsed. When the optical source is pulsed, the pulse has a range of from 1 picosecond to 10 picosecond micropulse and from 1 microsecond to 10 microsecond macropulse. Also, focusing of the optical energy occurs in an area in a range of 50 micrometers to 600 micrometers.

In certain embodiments, the present invention provides a method of exciting a target tissue comprising: (a) providing a laser to generate a laser beam having a wavelength in a range of from two micrometers to nine micrometers at a power output in a range of from 100 microjoules to 5 millijoules, having an area in a range of 50 micrometers to 600 micrometers, (b) providing a target tissue, (c) focusing the laser beam on the target tissue so that the target tissue conducts a nerve signal. It may be desired to pulse the light source. Although other pulse widths and durations may be used, a pulse can have a range of from 1 picosecond to 10 picosecond micropulse and from 1 microsecond to 10 microsecond macropulse.

The present invention also discloses a system used for stimulating neural tissue without damaging the neural tissue. The present invention discloses a method of stimulating neural tissue by providing an optical source to generate a beam of radiation having a wavelength which approximately corresponds to a valley for water absorption of a neural tissue, providing the neural tissue, and directing the beam of radiation at the neural tissue to be stimulated. Also disclosed is a method stimulating target tissue by providing a source capable of generating an optical energy having a wavelength in a range of from 0.9 micrometers to six micrometers at a fluence in a range of from 0.07 J/cm² to 25 J/cm², providing a target neural tissue, and focusing the optical energy on the target neural tissue so that action potentials are propagated. During this method, the source may be pulsed. Additionally, the target neural tissue can be mammalian neural tissue, or human neural tissue.

The methods disclosed herein may not damage neural tissue. Neural tissue may be irradiated at sub-ablative fluence, a wavelength of 4.5 micrometers with a fluence of 0.84 J/cm². No discernible damage may be caused at this fluence. At fluence levels of approximately 0.84 J/cm², levels that may induce clear potentials in a nerve, there may be no thermal damage as observed under light microscopy.

Energy is generally known to have three types of effects on tissue. While photothermal and photochemical effects on neural tissue have been widely studied, the third type of effect, photomechanical, appears to play a minor role with regard to neural tissue. Thus, modifications to the pulse parameters may be used to identify modifications to the photothermal and photochemical responses by the neural tissues.

Spot size of the laser beam may be reduced in size. By doing so a small portion of the target tissue may be selectively stimulated without disturbing the other elements of the target tissue. By doing so, this may be an effective way for an investigator to perform a functional identification of the target tissue. For example, a researcher would have the ability to map the different portions of a brain nucleus or cortical area to the specific muscular tissue they innervate, or the specific symptom results that they produce. For a clinician, this will serve as a tool to selectively identify the points of damage within a nerve or map subsections of the nerve.

### Regulation of Targeted Brain Regions

One aspect of this invention relates to the selection of brain regions. As has been noted, the brain contains thousands of individually named structures with different functions and anatomical locations. There are also hundreds of conditions that involve inappropriate functioning of areas of the brain. As a result, there are many hundreds of thousands of potential treatment targets, each involving the inappropriately functioning area(s) of the brain for the particular condition.

As has been disclosed, this invention provides for the regulation of discrete brain regions for use in the treatment of particular conditions associated with those conditions. Thus, by first selecting a region of interest based on a particular condition, various methods are provided for the regulation of that region of interest and hence the particular condition associated with it. For example, methods are provided that allow one to measure activity of one or more regions of interest associated with a particular condition; employ computer executable logic that takes the measured brain activity and determines parameters for use in stimulation of tissue using this invention. It should be recognized that the other various methods according to the present invention can be directed to any region of interest and thus can be applied to conditions associated with particular regions of interest.

A further aspect of the present invention relates to the localization of particular brain regions for use in the treatment of particular conditions. By knowing these brain regions, a device operator or subject may select and localize a region of interest.

FIG. 6-8 provide particular examples of brain regions that may be used as regions of interest for stimulation and regulation, particularly as noted in the columns labeled regions and coordinates. It is noted that the structures and coordinates shown in FIG. 6-8 should be understood to include either unilateral instances of these structures and positions in either hemisphere, or bilateral instances of these structures including both hemispheres. In addition, an effective method for the stimulation of a given neural region may be the stimulation to regulate a named anatomical target of one of the regions shown, rather than the location itself, using the anatomical target as the region of interest for stimulation. Therefore, the named anatomical targets of the regions described in FIGS. 6-8 may be used in stimulation for the purposes designated, rather than or in addition to the locations themselves.

A device operator may also use the coordinates provided in FIG. 6-8 as the center for a region of interest. These coordinates are presented in standard Talairach space. Therefore, before selection of a region of interest, these coordinates may be transformed into the coordinate frame of the subject being stimulated. The invention may then be used for the modulation of the selected region.

The regions designated in FIGS. 6-8 may be used as regions of interest for any of the embodiments of the invention disclosed herein. Specifically, these regions may be used as the targets for brain stimulation. In addition, it will be understood by one skilled in the art that there is some variability in the location of structures across subjects. The locations designated may be used as regions of interest for any of the embodiments of the invention disclosed herein, as may locations including these regions of interest, as may nearby locations, such as locations within 1, 2, 5, 10 cm from the described location.

Once the one or more regions of interest are identified and localized for the particular subject, and exemplar behaviors and/or stimuli may be identified to use in stimulating the one or more region of interest for the particular subject, stimulation of the one or more regions of interest can be performed according to the present invention.

### Regulation of Targeted Brain Regions for Treatment of Particular Conditions

In addition to the large number of brain regions that may be used as targets for stimulation, such as those listed in FIG. 6-8, there are also hundreds of conditions that involve inappropriate functioning of areas of the brain.

By associating a given condition with a particular brain region, and then by stimulating that particular brain region according to the present invention, treatment of the conditions can be achieved. Furthermore, some conditions relate to an injury or damage (such as from a stroke) to a given brain region. By knowing the location of the injury or damage, localizing a region of interest relative to the injury or damage, such as adjacent to the area of damaged tissue, stimulation of the regions can be performed. For example, in one embodiment, a method is provided according to the present invention comprising taking a subject having a condition, identifying one or more regions of interest for the subject where the treatment of those one or more regions would benefit the subject regarding the condition; and stimulating the one or more regions according to a method according to the present invention. Examples of particular conditions and associated regions of interest are provided in FIG. 6-8.

FIGS. 6-8 present combinations of brain regions of interest, and particular conditions for which those regions of interest may be appropriately used in stimulation. When a subject has been identified and screen who has a particular condition, one or more regions of interest may be selected from FIGS. 6-8 that is appropriate to the condition of the subject, and stimulation of the one or more regions of interest may be performed according to the present invention. It will be noted that some regions of interest are related to more than one condition, for instance, the nucleus basalis provides cholinergic innervation of the cerebral cortex, so it is involved in normal learning and plasticity, and it is also involved in the loss of memory associated with the decreased cholinergic functioning found in Alzheimer's disease. Similarly, the substantia nigra is a primary source of dopaminergic modulation, which has been repeatedly shown over many decades to be involved in both Parkinson's disease and schizophrenia. As another example, stimulation of the anterior cingulated cortex, and/or the rostral anterior cingulate cortex, may be used in the treatment of chronic pain.

As another example, subjects with Alzheimer's disease have decreased activity in the nucleus basalis of Meynert, due in part to neuronal degeneration. This decrease in activity in nucleus basalis is understood in the art to lead to a decrease in cholinergic activation of the cerebral cortex, with resulting memory and cognitive impairments. Once again, prior art has described electrical stimulation of the nucleus basalis as a means of overcoming certain effects of Alzheimer's disease. In one example of using the present invention, these subjects with Alzheimer's disease may be treated through stimulation that allows increase in the activity in the nucleus basalis. This may lead the nucleus basalis to release acetyl choline onto neurons in the cortex at a higher level than the diminished level found in the disease state.

As another example, subjects with Depression have decreased activation both in the serotonergic nuclei, and in certain cortical zones including frontal lobe regions. Subjects with depression and other psychological disorders such as social phobia may be treated by stimulation of serotonergic nuclei. These nuclei may release serotonin and increase its level to higher than the diminished level found in the disease state, as well as increase the activity level of certain target regions of serotonergic modulation, such as frontal cortical regions. Depression may also be address through stimulation of the subgenual cingulate using methods provided here.

As another example, subjects with chronic pain may be treated through the control of certain antinociceptive regions of the brain, as provided for in FIG. 6-8. Activation of these regions, which may include the periaqueductal gray, nucleus raphe magnus, insula, cingulate cortex, somatosensory cortex, medial thalamus, and dorsal horn of the spinal cord, may lead to a decrease in experienced pain. Subjects may be stimulated using one or more of these regions as a target tissue.

As another example, subjects with epilepsy have areas of the brain where excessive activation leads to seizures. This method provides for stimulation within or adjacent to epileptic tissue in order to disrupt or prevent an epileptic seizure. Seizure activity may be monitored through measurement of brain electrical activity, computations may be made to determine the presence or likely onset of a seizure, and this method may be used to produce stimuli to the epileptic tissue, to adjacent tissue, or to connected tissue that will block, prevent, or diminish the seizure.

### Regulation of Targeted Brain Regions for Neuromodulatory Effects

There are a large variety of areas in the brain that serve the primary role of releasing neuromodulatory agents, such as opioids, neuropeptides, acetylcholine, dopamine, norepinephrine, serotonin and other biologic amines, and others. Many of these compounds are the compounds mimicked by exogenously administered pharmacologic agents. The stimulation of particular brain regions may be used to stimulate the release of particular neuromodulatory agents that are released when those regions become active. For example, in one embodiment, a method is provided according to the present invention comprising: identifying one or more regions of interest that release neuromodulatory agents for a subject; and stimulating the one or more regions according to a method according to the present invention such that an amount of neuromodulatory agents released by the regions of interest is altered, preferably increased. Examples of particular release neuromodulatory agent releasing regions of interest are provided in FIGS. 6-8.

By associating a given condition with a neuromodulator, and then by stimulating that particular brain region according to the present invention, the release of that neuromodulator can be achieved. FIGS. 6-8 presents combinations of brain regions of interest, and particular neuromodulators for which those regions of interest may be appropriately used in stimulation. When a subject has been identified and screened who would be expected to benefit from the administration of a particular neuromodulatory substance, or from pharmacologic agents designed to mimic that neuromodulatory substance, one or more regions of interest may be selected from FIGS. 6-8 or from other brain regions that are appropriate to that neuromodulatory substance, and stimulation of the one or more regions of interest may be performed according to the present invention. The release of the neuromodulatory substance may then be monitored using methods for monitoring peripheral or central levels of a neuromodulator that are described in the literature, or using behavioral or symptom measures. Scanning methods such as PET may be used to measure the level of central neuromodulators released.

It is noted that sub-regions of neuromodulatory centers may also be controlled according to the present invention so that not all targets even of a single neuromodulatory center receive the same level of increased activation. This may allow a degree of specificity of the generation of internal release that may be even greater. It may also be possible to control multiple neuromodulatory areas together to produce combined effects.

As an example, subjects that would benefit from the use of serotonergic drugs such as citalopram, fluoxetine, fluvoxamine, paroxetine and sertraline, may be stimulated to activate brain regions that endogenously release serotonin, such as those described in FIGS. 6-8. Specifically, if a subject is stimulated to activate the raphe nucleus, this may lead to the release of serotonin.

### Regulation of Targeted Brain Regions for Plasticity and Learning

The present invention may also be used to enhance neuronal plasticity and learning. For example, in one embodiment, a method is provided according to the present invention comprising: identifying one or more regions of interest associated with neuronal plasticity and learning for a subject; and stimulating the one or more regions according to a method according to the present invention such that neuronal plasticity and learning for the subject is improved. Examples of particular neuronal plasticity and learning regions of interest are provided in FIGS. 6-8.

Several regions in the brain are known to be involved in controlling plasticity generally, including for example, those listed in FIGS. 6-8. Such regions may be selected and localized, for example the selection and localization may be carried out as described in section 4, and a subject is selected. The selection of subjects is as provided for in section 2, selecting subjects that will benefit from enhanced plasticity or learning of a particular task, or particular knowledge. Additional material may also be presented to the subject to guide the subject's learning. The invention may then be used for the stimulation of the region designated in FIGS. 6-8. The invention may also be used to stimulate an additional region of interest during the modulation of a region involved in enhanced plasticity, for the purpose of improving the stimulation and modulation of that additional region. By stimulating multiple regions a synergistic effect may be achieved. In addition, by repeated stimulation of multiple regions of the brain or targets, the activation of those to regions may become more greatly coupled through synaptic plasticity.

The regions associated with plasticity and learning have been shown to lead to increases in plasticity and learning when they are activated. A method is provided for enhancing plasticity and learning by increasing a level of activity in one or more of the regions designated in FIGS. 6-8 as being involved in plasticity and learning. This region may be selected as a region of interest for stimulation. This may constitute increasing the activity of one or more regions involved in plasticity or learning.

Use in Combination with Other Interventions

The methods described in this invention may be used in combination with a number of different additional methods, as described here.

Use in Combination with Pharmacology

It is recognized that the various methods according to the present invention may be performed in combination with pharmacologic intervention which may make such methods more effective.

Producing Brain Activation Similar to that Produced by Pharmacologic Agents

Stimulation may be used to replicate the activity provided by a pharmacologic agent. This would allow discontinuation of the drug use or reduction of the drug dosage. According to this variation, brain activity in selected regions is measured with and without the pharmacologic agent, and regions of interest are defined as regions with a selective difference in activation between these two conditions. Then, those identified regions of interest are targeted to be stimulated according to the present invention. This may also take place in combination with the provision of the pharmacologic agent, which may increase the efficacy of the pharmacologic agent, or decrease the necessary dose.

In the example case of Parkinson's disease, any pharmacologic agent that ameliorates Parkinson's disease symptoms may be used. Particular examples include, but are not limited to: L-dopa, pergolide, bromocryptine, promipexole and ropinirole. When a patient has been administered one of these agents and shows improved symptoms, brain activity may be measured in all or part of the brain. This measurement may take place using brain imaging such as fMRI or PET. This activity may be compared with activity in the absence of the agents, or when symptoms are worsened. The activity pattern measured during successful treatment with one of these agents, or the difference between the pattern measured during successful treatment and without successful treatment, may be used as a target activity pattern for stimulation.

Use in Combination with Device or Pharmacologic Testing

It is envisioned that the present invention may also be used to determine the likely long-term success outcome of a pharmacologic treatment, or to set appropriate dosage for that treatment, or to test the effectiveness of stimulation.

It is noted in regard to this section that the subject used here may not be human but rather may be another animal, such as a mouse, rabbit, cat, dog, monkey, sheep, pig, or cow that is to be used in testing. Because such animals do not have the cognitive ability of humans to receive and process instructions, it is recognized that the stimuli or instructions for behavior used will necessarily be limited to those stimuli or instructions for behavior that the animal can be effectively asked to perform or which the animal can be made to perform. For example, the stimulus may be an external stimulus such as a sound, a smell, a bright light, or a nociceptive stimulus, that is applied to the animal.

In order to test a pharmacologic agent, the methods provided here may be used to stimulate a target tissue in the presence of different concentrations of the pharmacologic agent, or in the absence of the pharmacologic agent, and the physiological response may be compared. For example, in a hippocampal slice preparation the methods disclosed here may be used to stimulate fibers synapsing onto a group of neurons whose activation is measured electrically, including glutamatergic synapsing fibers. The electrical potential in a target cell or population of cells may be recorded that results from light stimulation of the fibers using this method. Then, a drug such as a potential glutamate antagonist may be applied, for example in fluid 35 through catheter 34. The electrical potential may be compared in the presence and absence of the drug to determine the drugs effect. Similarly, methods may be used to monitor the effect of a drug on a physiological response induced using light stimulation in vivo.

### Localization of Neuronal Function, Especially for Neurosurgery

The present invention may also be used to localize within the brain the correlates of certain psychological or neurological functions. For example, through stimulation it may be possible to determine the areas that underlie particular psychological or neurologic functions. If the physiological criteria selected are stimulation correlated with a particular behavioral outcome, then the brain regions engaged during stimulation and performance of this task are determined. This can be used as a method for determining where areas are located. This may be useful in neurosurgery, such as for the sparing of regions or hemisphere involved in language, and regions involved in motor control.

### Three Dimensional Light Patterns

Light stimuli may be shaped to produce 3 dimensional patterns. This may be accomplished through lenses, multiple beams which converge on a given location, or holography. One or more lenses may be used to focus light upon a target location, thereby achieving specificity of stimulation location. In the case of holography, a hologram may be used that produces laser stimulation at a 3 dimensional array of locations, defined by the hologram. For example, if a hologram is created that corresponds to an image of a neural structure, and this hologram is used with applied laser light, this allows the laser light to produce the greatest activation in the region of the intended neural structure.

### Types of Light Sources

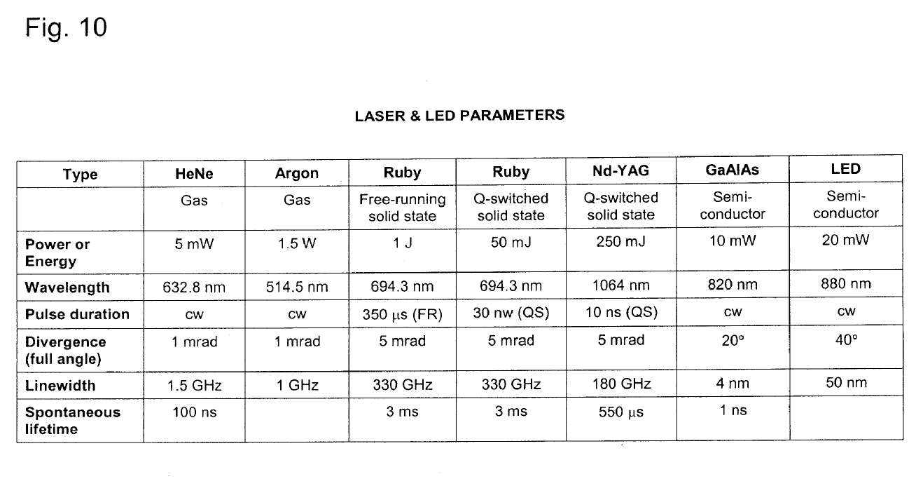

In addition to the types of sources already described, light sources used in combination with this invention may include, but are not limited to, those summarized in FIG. 10.

### Nerve Cuff Light-Emitter

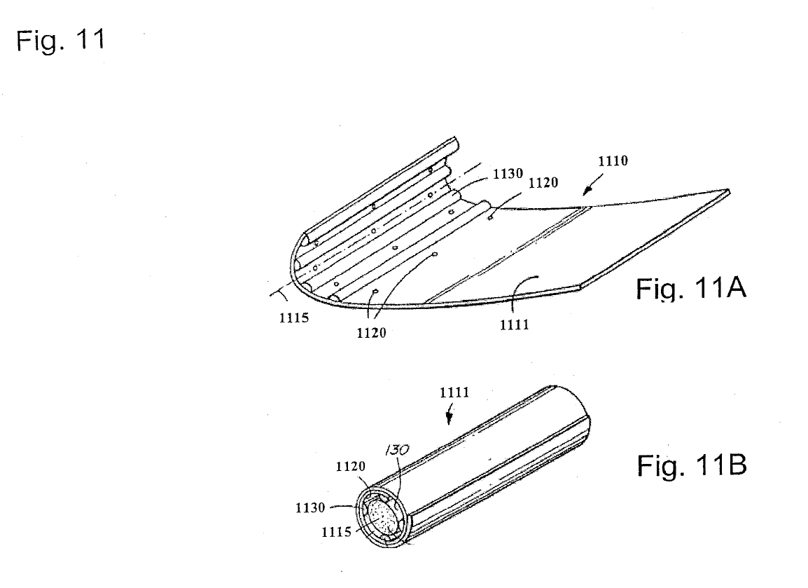

In one embodiment, light-emitters may be fashioned to be positioned around a nerve fiber or nerve bundle to form a cuff, as shown in FIG. 11. This method may be adapted from nerve cuff electrical stimulation methods, such as provided in U.S. Pat. No. 5,824,027. Electrodes used for nerve stimulation may be replaced by light emitters for nerve stimulation. Similar to the use of nerve cuffs using electrical stimulation, the current invention provides for a light-emitter surrounding an element of tissue so as to stimulate the tissue from multiple angles. The light-emitter may provide light at multiple locations adjacent to the tissue being stimulated, and at multiple points along the length of the tissue, e.g. multiple points along a nerve. In one embodiment, separate elements of the target tissue such as separate groups of neurons in a peripheral nerve or brain nucleus may be individually controlled through individually controlling multiple stimulating light-emitters, each adjacent to an element of the target tissue.

FIGS. 11A and 11B illustrate a nerve cuff 1110 according to a further alternative embodiment of the invention. Nerve cuff 1110 comprises a self-curling sheet 1111 biased to curl upon itself around an axis 1115 to form an annular nerve cuff having a bore. A nerve can be inserted through bore by unrolling sheet 1111 and then permitting sheet 1111 to curl around a nerve in a controlled manner. Nerve cuffs of this general type are described in Naples et al., U.S. Pat. No. 4,602,604. A plurality of rounded ridges 1130 extend along sheet 1111 in a generally longitudinal direction.

When nerve cuff 1110 is in its curled up configuration, as shown in FIG. 11B, ridges 1130 project into bore. One or more light emitters 1120 suitable for nerve stimulation and/or recording may be provided on sheet 1111 between ridges 1130. In the alternative, fluid conduction means, such as tubes, may be provided to conduct fluids into or out of bore.

### Vagal Nerve Stimulation

In one embodiment, light stimulation may be used to stimulate the vagus nerve, replacing electrical stimulation. Vagal nerve stimulation may be used as a replacement for electrical stimulation in all known and potential future uses for vagal nerve stimulation, including Epilepsy, Depression. Vagal nerve stimulation is described in Schachter S C. “Vagus nerve stimulation: current status and clinical applications” Expert Opin Investig Drugs. 1997 October; 6(10):1327-35 and references cited therein.

### Tissue Measurement

The light conductor provided in this invention may be used for the optical measurement of tissue activity. For example, blood oxygenation within the subject may be measured through measurements of the spectrum of light observed through the light conductor. Nerve or neuron activation may also be measured using optical measurements through a light conductor.

### Characterization of Brain Regions

An additional example of this invention relates to the characterization of brain regions of unknown or only partially known function. Through the use of this invention, it is possible to characterize the functioning of a localized brain region of interest. In this example, a brain region to be characterized is selected as a region of interest. A procedure is laid out for the stimulation of brain regions of interest. This knowledge of the characterization of a brain region may be used for a variety of purposes. For example, this new knowledge may be used to design treatments involving the characterized brain region of interest. These treatments may include pharmacological treatments, surgical treatments, electrical stimulation treatments, or other treatments. The knowledge of the characterization of a brain region may be used for diagnostic purposes as well. For instance, if it has been determined that a brain region of interest is implicated in a condition, such as a disease, then using the stimuli or behaviors determined to engage that brain region may be used as a diagnostic for whether a subject has that condition, and the extent or severity of the condition. These stimuli or behaviors determined to engage the brain region may also be used in conjunction with a pharmacologic treatment as a means for determining the effect of the pharmacologic treatment on the activation observed in the brain region of interest in the presence and absence of the pharmacologic treatment. This may be used as a means for assessing the pharmacologic treatment.

What is claimed is:

1. A system for stimulating target tissue comprising:
a light source for providing stimulation pulses;
an implantable light conducting lead coupled to said light source adapted for stimulation of a predetermined site in a subject.

2. The system, as claimed in claim 1, wherein said light conducting lead is an optical fiber.

3. The system, as claimed in claim 1, wherein said light source is a laser.

4. The system, as claimed in claim 2, wherein said light source is implantable.

5. A method of treating a disorder comprising:
implanting at least one light-emitter coupled to a light source such that it is in communication with at least one predetermined site in the nervous system of a body;
stimulating said at least one predetermined site in said nervous system of said body using said at least one light-emitter.

6. The method of claimed in claim 5, wherein said disorder is Parkinson's disease, Alzheimer's disease, depression, or epilepsy.

7. The method of claim 5 further comprising the step of
regulating at least one parameter of said step of stimulating, said at least one parameter being selected from the group consisting of pulse width, pulse frequency, and pulse amplitude.

8. A method for treating a disorder in a patient comprising the steps of:
surgically implanting a light-emitter into a brain of a patient wherein said light emitter is coupled to a light source and a signal generator operating said light source; and
operating said signal generator to stimulate a predetermined treatment site in said brain.
